SEQUENCE LISTING (38 sequences)

SEQ ID NO: 1 - DNA, 15 bp, Artificial Sequence
  Fully synthetic
gcagctcaagaattc

SEQ ID NO: 2 - DNA, 24 bp, Artificial Sequence
  Fully synthetic
ccctcgagggaggctttgtttcaa

SEQ ID NO: 3 - protein, 4 aa, Artificial Sequence
  Fully synthetic
RKRK

SEQ ID NO: 4 - DNA, 12 bp, Artificial Sequence
  Fully synthetic
aggaagaggaag

SEQ ID NO: 5 - protein, 4 aa, Artificial Sequence
  Fully synthetic
KRKR

SEQ ID NO: 6 - DNA, 12 bp, Artificial Sequence
  Fully synthetic
aagaggaagagg

SEQ ID NO: 7 - protein, 4 aa, Artificial Sequence
  Fully synthetic
RRKK

SEQ ID NO: 8 - DNA, 12 bp, Artificial Sequence
  Fully synthetic
aggaggaagaag

SEQ ID NO: 9 - protein, 4 aa, Artificial Sequence
  Fully synthetic
KKRR

SEQ ID NO: 10 - DNA, 12 bp, Artificial Sequence
  Fully synthetic
aagaagaggagg

SEQ ID NO: 11 - protein, 4 aa, Artificial Sequence
  Fully synthetic
KRRK

SEQ ID NO: 12 - DNA, 12 bp, Artificial Sequence
  Fully synthetic
aagaggaggaag

SEQ ID NO: 13 - protein, 4 aa, Artificial Sequence
  Fully synthetic
RKKR

SEQ ID NO: 14 - DNA, 12 bp, Artificial Sequence
  Fully synthetic
aggaagaagagg

SEQ ID NO: 15 - protein, 5 aa, Artificial Sequence
  Fully synthetic
RRHKK

SEQ ID NO: 16 - DNA, 15 bp, Artificial Sequence
  Fully synthetic
aggaggcacaagaag

SEQ ID NO: 17 - protein, 6 aa, Artificial Sequence
  Fully synthetic
KHRKHR

SEQ ID NO: 18 - DNA, 18 bp, Artificial Sequence
  Fully synthetic
aagcacaggaagcacagg

SEQ ID NO: 19 - protein, 6 aa, Artificial Sequence
  Fully synthetic
KHRCKP

SEQ ID NO: 20 - DNA, 18 bp, Artificial Sequence
  Fully synthetic
aagcacaggtgtaagcca

SEQ ID NO: 21 - protein, 6 aa, Artificial Sequence
  Fully synthetic
RRHKMK

SEQ ID NO: 22 - DNA, 18 bp, Artificial Sequence
  Fully synthetic
aggaggcacaagatgaag

SEQ ID NO: 23 - protein, 6 aa, Artificial Sequence
  Fully synthetic
HRKCRK

SEQ ID NO: 24 - DNA, 18 bp, Artificial Sequence
  Fully synthetic
cacaggaagtgtaggaag

SEQ ID NO: 25 - protein, 7 aa, Artificial Sequence
  Fully synthetic
KRWRKHR

SEQ ID NO: 26 - DNA, 21 bp, Artificial Sequence
  Fully synthetic
aagaggtggaggaagcacagg

SEQ ID NO: 27 - protein, 8 aa, Artificial Sequence
  Fully synthetic
HKGRKCRV

SEQ ID NO: 28 - DNA, 24 bp, Artificial Sequence
  Fully synthetic
cacaagggaaggaagtgtagggtg

SEQ ID NO: 29 - protein, 9 aa, Artificial Sequence
  Fully synthetic
KRWHKMRKH

SEQ ID NO: 30 - DNA, 27 bp, Artificial Sequence
  Fully synthetic
aagaggtggcacaagatgaggaagcac

SEQ ID NO: 31 - protein, 11 aa, Artificial Sequence
  Fully synthetic
HFWRQCALAMK

SEQ ID NO: 32 - DNA, 27 bp, Artificial Sequence
  Fully synthetic
cacttctggcgacaatgtgctatgaag

SEQ ID NO: 33 - protein, 9 aa, Artificial Sequence
  Fully synthetic
YFPRHQKWK

SEQ ID NO: 34 - DNA, 27 bp, Artificial Sequence
  Fully synthetic
tacttccctcgtcatcaaaagtggaag

SEQ ID NO: 35 - protein, 9 aa, Artificial Sequence
  Fully synthetic
WKYRQISTC

SEQ ID NO: 36 - DNA, 27 bp, Artificial Sequence
  Fully synthetic
tggaagtaccgccagatcagcacctgc

SEQ ID NO: 37 - protein, 10 aa, Artificial Sequence
  Fully synthetic
RKHKMRKCHK

SEQ ID NO: 38 - DNA, 30 bp, Artificial Sequence
  Fully synthetic
aggaagcacaagatgaggaagtgtcacaag

(19) United States
(12) Patent Application Publication
(10) Pub. No.: US 2019/0284538 A1
(43) Pub. Date: Sep. 19, 2019

(54) RECOMBINANT ONCOLYTIC VIRUS, SYNTHETIC DNA SEQUENCE, AND APPLICATION THEREOF

(71) Applicant: Wuhan Boweid Biotechnology Co., Ltd., Wuhan (CN)

(72) Inventor: Ligang Cai, Wuhan (CN)

(21) Appl. No.: 16/352,806

(22) Filed: Mar. 13, 2019

Related U.S. Application Data
(63) Continuation-in-part of application No. 62/643,166, filed on Mar. 14, 2018.

(51) Int. Cl. C12N 7/00; A61K 35/768; C07K 5/10; C07K 7/06; A61P 35/00

(52) U.S. Cl. CPC C12N 7/00; A61K 35/768; C07K 5/10; C12N 2770/32332; A61P 35/00; C12N 2770/32321; C07K 7/06

(57) ABSTRACT

The disclosure provides a recombinant oncolytic virus, a synthetic DNA sequence and applications of the virus. The recombinant oncolytic virus includes a genome and an exogenous DNA sequence inserted in the genome. The exogenous DNA sequence adapts to express a basic peptide fragment, to increase the environmental pH in a host infected by the recombinant oncolytic virus. The basic peptide fragment includes more than 60% of basic amino acids. The recombinant oncolytic virus and the synthetic DNA sequence of the disclosure are used to prepare an anti-tumor drug.

### CROSS-REFERENCE TO RELATED APPLICATIONS

This application is a continuation-in-part of U.S. Provisional Application No. 62/643,166 filed Mar. 14, 2018, the contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND

The disclosure relates to a recombinant oncolytic virus, a synthetic DNA sequence, and application thereof.

Oncolytic viruses are viruses that preferentially infect and kill cancer cells. Wild-type oncolytic viruses are toxic. When an organism is infected with a wild-type oncolytic virus, systemic symptoms such as fever and chills occur.

### SUMMARY

Disclosed is a recombinant oncolytic virus, a synthetic DNA sequence, and methods of using the same. The disclosure aims at inhibiting tumor cells by providing a recombinant oncolytic virus capable of modifying the microenvironment of the tumor.

To achieve the above objective, according to one embodiment of the invention, there is provided a recombinant oncolytic virus, comprising: an oncolytic virus genome and an exogenous DNA sequence inserted in the oncolytic virus genome, the exogenous DNA sequence being adapted to express a basic peptide fragment, to increase environmental pH in a host infected by the recombinant oncolytic virus.

The oncolytic virus genome can be a genome of herpes virus, Coxsackie viruses, adenovirus, cowpox virus, measles virus, poliomyelitis virus, retroviruses, reovirus, respiratory syncytial virus, parvovirus H1, vesicular stomatitis virus, or Newcastle disease virus, and preferably adenovirus, Newcastle disease virus, or Coxsackie virus.

The environmental pH in the host infected by the recombinant oncolytic virus can be increased by about 0.4 to about 0.6.

The basic peptide fragment can comprise 4 to 10 amino acids.

The basic peptide fragment can comprise more than 60% of basic amino acids.

The basic peptide fragment can comprise more than 80% of the basic amino acids.

The basic amino acids can be selected from Arginine, Lysine, and Histidine.

The basic amino acids can be selected from Arginine and Lysine.

The basic peptide fragment can be selected from:

(SEQ ID NO. 3)

Arg-Lys-Arg-Lys;

(SEQ ID NO. 5)

Lys-Arg-Lys-Arg;

(SEQ ID NO. 7)

Arg-Arg-Lys-Lys;

(SEQ ID NO. 9)

Lys-Lys-Arg-Arg;

(SEQ ID NO. 11)

Lys-Arg-Arg-Lys;

(SEQ ID NO. 13)

Arg-Lys-Lys-Arg;

(SEQ ID NO. 15)

Arg-Arg-His-Lys-Lys;

(SEQ ID NO. 17)

Lys-His-Arg-Lys-His-Arg;

(SEQ ID NO. 19)

Lys-His-Arg-Cys-Lys-Pro;

(SEQ ID NO. 21)

Arg-Arg-His-Lys-Met-Lys;

(SEQ ID NO. 23)

His-Arg-Lys-Cys-Arg-Lys;

(SEQ ID NO. 25)

Lys-Arg-Trp-Arg-Lys-His-Arg;

(SEQ ID NO. 27)

His-Lys-Gly-Arg-Lys-Cys-Arg-Val;

(SEQ ID NO. 29)

Lys-Arg-Trp-His-Lys-Met-Arg-Lys-His;

(SEQ ID NO. 31)

His-Phe-Trp-Arg-Gln-Cys-Ala-Met-Lys;

(SEQ ID NO. 33)

Tyr-Phe-Pro-Arg-His-Gln-Lys-Trp-Lys;

(SEQ ID NO. 35)

Trp-Lys-Tyr-Arg-Gln-Ile-Ser-Thr-Cys;
and

(SEQ ID NO. 37)

Arg-Lys-His-Lys-Met-Arg-Lys-Cys-His-Lys.

With regard to the recombinant oncolytic virus, the recombinant oncolytic virus can be Coxsackie virus B3 strain.

With regard to the recombinant oncolytic virus, the basic peptide fragment can be selected from:

(SEQ ID NO. 11)

Lys-Arg-Arg-Lys;

(SEQ ID NO. 29)

Lys-Arg-Trp-His-Lys-Met-Arg-Lys-His;
and

(SEQ ID NO. 31)

His-Phe-Trp-Arg-Gln-Cys-Ala-Met-Lys.

With regard to the recombinant oncolytic virus, the recombinant oncolytic virus can be a variant attenuated Coxsackie virus B3 strain comprising base mutations of T97C, G1180A, T1654C, T1756C, G2276A, A2685C, G2690A, C3120A, A3231G, G4327A, T5088C, A5270G, C7026T, and/or G7192A.

The exogenous DNA sequence can be inserted onto a pVAX1 vector.

With regard to the recombinant oncolytic virus, the basic peptide fragment can be

(SEQ ID NO. 29)

Lys-Arg-Trp-His-Lys-Met-Arg-Lys-His.

According to another aspect of the disclosure, the disclosure provides an application of the abovementioned recombinant oncolytic virus for preparation of an anti-tumor drug.

The anti-tumor drug can be an anti-solid tumor drug.

The anti-solid tumor drug can be used for treating respiratory tract tumors, gastrointestinal tumors, endocrine tumors, or gynecological tumors.

According to another aspect of the disclosure, an anti-tumor drug is provided, which comprises the recombinant oncolytic virus provided in the disclosure.

The anti-tumor drug can further comprise a checkpoint inhibitor.

According to another aspect of the disclosure, there provided is a method for treating a malignant tumor, the method comprising administering the anti-tumor drug intravenously or locally to a patient in need thereof.

The malignant tumor can be a solid tumor.

The malignant tumor can be respiratory tract tumor, gastrointestinal tumor, endocrine tumor, or gynecological tumor.

According to another aspect of the disclosure, a synthetic DNA sequence is provided, which is adapted to express a basic peptide fragment comprising more than 60% of basic amino acids.

With regard to the synthetic DNA sequence, the basic peptide fragment can comprise more than 80% of the basic amino acids.

The basic amino acids can be selected from Arginine, Lysine, and Histidine.

With regard to the synthetic DNA sequence, the basic amino acids can be selected from Arginine, and Lysine.

With regard to the synthetic DNA sequence, the basic peptide fragment can be selected from:

(SEQ ID NO. 3)

Arg-Lys-Arg-Lys;

(SEQ ID NO. 5)

Lys-Arg-Lys-Arg;

(SEQ ID NO. 7)

Arg-Arg-Lys-Lys;

(SEQ ID NO. 9)

Lys-Lys-Arg-Arg;

(SEQ ID NO. 11)

Lys-Arg-Arg-Lys;

(SEQ ID NO. 13)

Arg-Lys-Lys-Arg;

(SEQ ID NO. 15)

Arg-Arg-His-Lys-Lys;

(SEQ ID NO. 17)

Lys-His-Arg-Lys-His-Arg;

(SEQ ID NO. 19)

Lys-His-Arg-Cys-Lys-Pro;

(SEQ ID NO. 21)

Arg-Arg-His-Lys-Met-Lys;

(SEQ ID NO. 23)

His-Arg-Lys-Cys-Arg-Lys;

(SEQ ID NO. 25)

Lys-Arg-Trp-Arg-Lys-His-Arg;

(SEQ ID NO. 27)

His-Lys-Gly-Arg-Lys-Cys-Arg-Val;

(SEQ ID NO. 29)

Lys-Arg-Trp-His-Lys-Met-Arg-Lys-His;

(SEQ ID NO. 31)

His-Phe-Trp-Arg-Gln-Cys-Ala-Met-Lys;

(SEQ ID NO. 33)

Tyr-Phe-Pro-Arg-His-Gln-Lys-Trp-Lys;

(SEQ ID NO. 35)

Trp-Lys-Tyr-Arg-Gln-Ile-Ser-Thr-Cys;
and

(SEQ ID NO. 37)

Arg-Lys-His-Lys-Met-Arg-Lys-Cys-His-Lys.

With regard to the synthetic DNA sequence, the basic peptide fragment can be selected from:

(SEQ ID NO. 11)

Lys-Arg-Arg-Lys;

(SEQ ID NO. 29)

Lys-Arg-Trp-His-Lys-Met-Arg-Lys-His;
and

(SEQ ID NO. 31)

His-Phe-Trp-Arg-Gln-Cys-Ala-Met-Lys.

Advantages of the embodiments of the disclosure include:

(1) The recombinant oncolytic virus provided in the disclosure modifies the interstitial pH in the tumor focus, so as to affect the microenvironment where the tumor cells grow and inhibit the tumor growth. The recombinant oncolytic virus has a broad-spectrum anti-tumor effect and has good application prospects when used during preparation of anti-tumor drugs. The recombinant oncolytic virus acts on the microenvironment where the tumor grows instead of the tumor cells themselves. The recombinant oncolytic virus proliferates with the proliferation of the malignant tumor cells and continuously express the basic polypeptide, so the anti-tumor effect is cumulative and automatically adapts to the development of the tumors without over-treatment.

In a preferred embodiment, the recombinant oncolytic virus provided in the disclosure may be obtained from various known oncolytic viruses. Because the expression of the basic peptide fragment acts on the cellular microenvironment, which does not conflict with the mechanisms of action of recombinant oncolytic viruses that express specific genes killing or inhibiting tumor cells, the inhibition effects are mutually beneficial to each other, and the anti-tumor effect is significant.

For example, Coxsackie CVB 3 is used in combination with a gene expressing a 4-peptide or 9-peptide fragment having lysine at the N-terminus, which can modify the interstitial acid-base environment in the tumor focus and has excellent anti-solid tumor effect. Moreover, the toxicity is low, and the adverse effect is small, causing only a mild fever response. In addition, Coxsackie virus is an RNA virus, which will not integrate into the host cells and causes no risk of transcription.

(2) The compositions for treating tumors provided in the disclosure are suitable for intravenous administration due to their safety, precision of targeting, high specificity, and low toxicity.

(3) The exogenous DNA sequence encoding the basic peptide fragment provided in the disclosure expresses the basic peptide fragment to change the pH in a cellular microenvironment and inhibits the growth of tumor cells.

### BRIEF DESCRIPTION OF THE DRAWINGS

FIG. 1 is a schematic gene map of Coxsackie virus carrying a eukaryotic expression vector pVAX1 provided in an example of the disclosure;

FIG. 2 is a schematic gene map of Coxsackie virus carrying a eukaryotic expression vector pVAX1 in which a synthetic DNA sequence is inserted provided in an example of the disclosure;

FIG. 3 is a simplified process chart of virus purification provided in an example of the disclosure;

FIG. 4 is a tumor volume curve provided in Example 20 of the disclosure;

FIG. 5 compares the tissue sections provided in Example 20 of the disclosure;

FIG. 6 is a tumor volume curve provided in Example 21 of the disclosure;

FIG. 7 is a tumor volume curve provided in Example 22 of the disclosure;

FIG. 8 is a tumor volume curve provided in Example 23 of the disclosure;

FIG. 9 shows the results of microscopic examination provided in Example 24 of the disclosure; and

FIG. 10 compares the pH measurements provided in Example 24 of the disclosure.

### DETAILED DESCRIPTION OF THE EMBODIMENTS

For further illustrating the invention, experiments detailing a recombinant oncolytic virus, a basic peptide fragment and applications thereof are described below.

The disclosure provides a recombinant oncolytic virus, which comprises an exogenous DNA sequence inserted in its genome, and expresses the basic peptide fragment in a physiological process, such that the environmental pH in a host infected therewith is increased by about 0.4 to 0.6. After infection with the recombinant oncolytic virus comprising the exogenous gene, the basic peptide fragment is highly expressed thus changing the microenvironment of the tumor tissue, such that the tumor tissues are inhibited and killed.

The recombinant oncolytic virus can be herpes viruses, Coxsackie viruses, adenovirus, cowpox virus, measles virus, poliomyelitis virus, retroviruses, reovirus, respiratory syncytial virus, parvovirus H1, vesicular stomatitis virus, or Newcastle disease virus. The recombinant oncolytic virus is preferably recombinant oncolytic viruses that deactivate or delete specific genes in the target cells, for example, adenovirus, Newcastle disease virus or Coxsackie viruses.

The basic peptide fragment comprises 4 to 10 amino acids, in which the basic amino acids account for more than 60%, and preferably more than 80%. The basic amino acids are selected from Arginine, Lysine, or Histidine, and preferably from Arginine or Lysine. The basic peptide fragment has an N-terminal amino acid that is Lysine optimally.

The basic peptide fragment is selected from:

(SEQ ID NO. 3)

Arg-Lys-Arg-Lys;

(SEQ ID NO. 5)

Lys-Arg-Lys-Arg;

(SEQ ID NO. 7)

Arg-Arg-Lys-Lys;

(SEQ ID NO. 9)

Lys-Lys-Arg-Arg;

(SEQ ID NO. 11)

Lys-Arg-Arg-Lys;

(SEQ ID NO. 13)

Arg-Lys-Lys-Arg;

(SEQ ID NO. 15)

Arg-Arg-His-Lys-Lys;

(SEQ ID NO. 17)

Lys-His-Arg-Lys-His-Arg;

(SEQ ID NO. 19)

Lys-His-Arg-Cys-Lys-Pro;

(SEQ ID NO. 21)

Arg-Arg-His-Lys-Met-Lys;

(SEQ ID NO. 23)

His-Arg-Lys-Cys-Arg-Lys;

(SEQ ID NO. 25)

Lys-Arg-Trp-Arg-Lys-His-Arg;

(SEQ ID NO. 27)

His-Lys-Gly-Arg-Lys-Cys-Arg-Val;

(SEQ ID NO. 29)

Lys-Arg-Trp-His-Lys-Met-Arg-Lys-His;

(SEQ ID NO. 31)

His-Phe-Trp-Arg-Gln-Cys-Ala-Met-Lys;

(SEQ ID NO. 33)

Tyr-Phe-Pro-Arg-His-Gln-Lys-Trp-Lys;

(SEQ ID NO. 35)

Trp-Lys-Tyr-Arg-Gln-Ile-Ser-Thr-Cys;
and

(SEQ ID NO. 37)

Arg-Lys-His-Lys-Met-Arg-Lys-Cys-His-Lys

In a preferred embodiment, Coxsackie virus, particularly an attenuated variant strain of Coxsackie virus is used. In a pVAX1 vector constructed to have the viral genome, a gene is inserted that expresses a basic peptide fragment selected from:

(SEQ ID NO. 5)

Lys-Arg-Lys-Arg;

(SEQ ID NO. 29)

Lys-Arg-Trp-His-Lys-Met-Arg-Lys-His;
and

(SEQ ID NO. 31)

His-Phe-Trp-Arg-Gln-Cys-Ala-Met-Lys.

In a further preferred embodiment, the basic peptide fragment is selected from

(SEQ ID NO. 11)

Lys-Arg-Arg-Lys;
and

(SEQ ID NO. 29)

Lys-Arg-Trp-His-Lys-Met-Arg-Lys-His.

When the Coxsackie virus CVB 3 strain is used, the exogenous peptide fragment is Lys-Arg-Trp-His-Lys-Met-Arg-Lys-His (SEQ ID NO. 29), so excellent tumor inhibition effect and good safety are exhibited.

The Coxsackie virus CVB 3 is preferably the variant attenuated Coxsackie virus B3 strain comprising base mutations of T97C, G1180A, T1654C, T1756C, G2276A, A2685C, G2690A, C3120A, A3231G, G4327A, T5088C, A5270G, C7026T, and/or G7192A. The sequence encoding the basic peptide fragment is inserted onto the pVAX1 vector.

The DNA sequence encoding the basic peptide fragment is inserted between the 5′UTR and VP4 of the recombinant vector.

Application of the recombinant oncolytic virus provided in the disclosure in the preparation of anti-tumor drug, particularly anti-solid tumor drugs, for examples, drugs for treating respiratory tract tumors, gastrointestinal tumors, endocrine tumors, or gynecological tumors, is further provided in the disclosure. The recombinant oncolytic virus provided in the invention, upon reaching the focal area, express the basic peptide fragment by making use of the targeting and replication ability of the virus, based on the severity of the tumor. It is experimentally confirmed that the basic peptide fragment changes the pH in the microenvironment where tumor cells resides, including the intracellular and intercellular environment. This change brings a series of comprehensive impacts on the metabolism of tumor cells, and finally results in an obvious tumor suppression effect. Therefore, the existing recombinant oncolytic viruses, in theory, can be genetically modified to carry the exogenous synthetic DNA sequence, so that in addition to the original inhibition on or killing of tumor cells, an inhibitory effect is produced by changing the microenvironment where the tumor cells grow. The two effects are synergistic, so as to inhibit the tumor more effectively. The recombinant oncolytic virus provided in the disclosure exerts an anti-tumor effect by impacting the microenvironment where the tumor cells grow, and thus has a more pronounced inhibitory effect on the solid tumor in which the tumor cells are concentrated. At the same time, for the recombinant oncolytic virus that causes serious physiological reaction, the dosage and dosage range of the virus used is reduced due to the superimposed tumor suppressive effect, so the physiological adverse effect resulting therefrom is lowered, the application of the recombinant oncolytic virus is widened and the safety of the recombinant oncolytic virus is improved.

The disclosure provides an anti-tumor drug, which comprises the recombinant oncolytic virus provided in the disclosure. Preferably, the anti-tumor drug further comprises an immune checkpoint inhibitor. The drug is administered intravenously or topically to the focus, thus exerting a good tumor suppressive effect. The immune checkpoint inhibitor (PD-1, PD-L1, and CTLA4) is not highly responsive to solid tumors (except melanoma), possibly due to the insufficient stimulation to the immune system in the patients with solid tumors after the release of the immune system by the checkpoint inhibitor. In the disclosure, the recombinant oncolytic virus and the immune checkpoint inhibitor synergize, thereby significantly enhancing the killing effect of the immune system for solid tumors, increasing the local infiltration of immune cells into the tumors, and up-regulating PD-L1. Particularly, the recombinant CVB3 virus induces the local production of specific and nonspecific immune responses in the tumor, such as exposure of calreticulin (CRT), translocation of ATP, and intracellular translocation of HGMB1 (Extracellular High Mobility Group Box 1). As the recombinant oncolytic virus proliferates, the IFN and/or cytokines are induced to produce activated NK and DC cells, whereby various mature DCs and cytotoxic CD107a+ NK cells are facilitate to enter the tumor site, resulting in the changes in the immune cellular profile in the tumor microenvironment and thus restoring the inherent antitumor immunity of an organism. The synergistic effect between the recombinant Coxsackie virus and the immune checkpoint inhibitor provided in the disclosure is particularly obvious.

The disclosure further provides a synthetic DNA sequence that expresses a basic peptide fragment. The basic amino acid in the basic peptide fragment has a content of more than 60% and preferably more than 80%. The basic amino acid is selected from Arginine, Lysine or Histidine, and preferably from Arginine or Lysine. The basic peptide fragment preferably has a N-terminal amino acid that is Lysine.

The basic peptide fragment encoded by the synthetic DNA sequence is elected from:

(SEQ ID NO. 3)

Arg-Lys-Arg-Lys;

(SEQ ID NO. 5)

Lys-Arg-Lys-Arg;

(SEQ ID NO. 7)

Arg-Arg-Lys-Lys;

(SEQ ID NO. 9)

Lys-Lys-Arg-Arg;

(SEQ ID NO. 11)

Lys-Arg-Arg-Lys;

(SEQ ID NO. 13)

Arg-Lys-Lys-Arg;

(SEQ ID NO. 15)

Arg-Arg-His-Lys-Lys;

(SEQ ID NO. 17)

Lys-His-Arg-Lys-His-Arg;

(SEQ ID NO. 19)

Lys-His-Arg-Cys-Lys-Pro;

(SEQ ID NO. 21)

Arg-Arg-His-Lys-Met-Lys;

(SEQ ID NO. 23)

His-Arg-Lys-Cys-Arg-Lys;

(SEQ ID NO. 25)

Lys-Arg-Trp-Arg-Lys-His-Arg;

(SEQ ID NO. 27)

His-Lys-Gly-Arg-Lys-Cys-Arg-Val;

(SEQ ID NO. 29)

Lys-Arg-Trp-His-Lys-Met-Arg-Lys-His;

(SEQ ID NO. 31)

His-Phe-Trp-Arg-Gln-Cys-Ala-Met-Lys;

(SEQ ID NO. 33)

Tyr-Phe-Pro-Arg-His-Gln-Lys-Trp-Lys;

(SEQ ID NO. 35)

Trp-Lys-Tyr-Arg-Gln-Ile-Ser-Thr-Cys;
and

(SEQ ID NO. 37)

Arg-Lys-His-Lys-Met-Arg-Lys-Cys-His-Lys.

The basic peptide fragment preferably has a sequence of:

(SEQ ID NO. 11)

Lys-Arg-Arg-Lys;

(SEQ ID NO. 29)

Lys-Arg-Trp-His-Lys-Met-Arg-Lys-His;
and

(SEQ ID NO. 31)

His-Phe-Trp-Arg-Gln-Cys-Ala-Met-Lys.

### Examples 1 to 18: Recombinant Coxsackie Viruses Having a Synthetic DNA Sequence Inserted in Their Genome

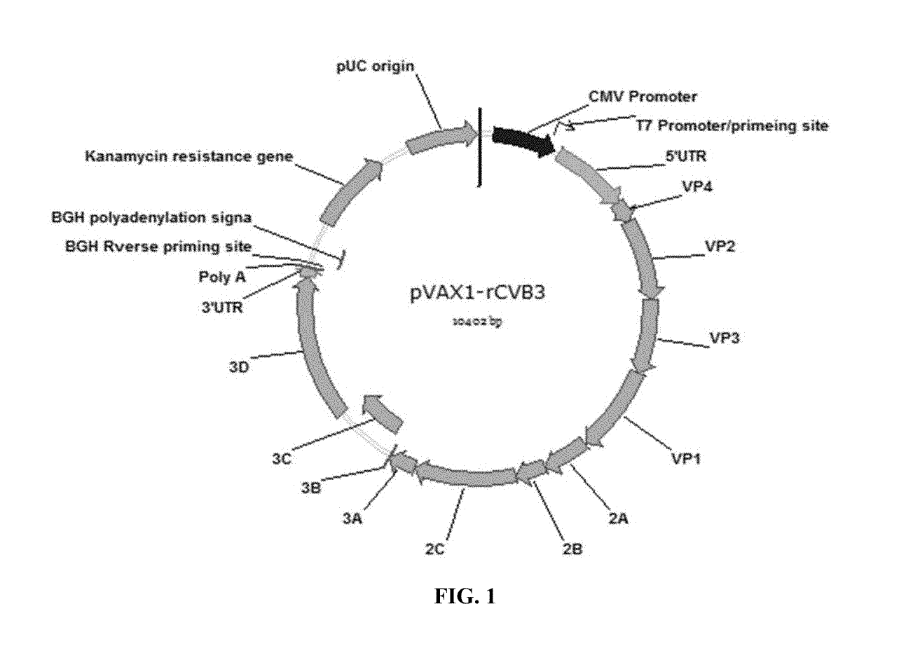

The whole genome sequence of the Coxsackie B3 nancy strain is as shown in GeneBank ID: JX312064.1. The recombinant Coxsackie virus strain comprises the base mutations of T97C, G1180A, T1654C, T1756C, G2276A, A2685C, G2690A, C3120A, A3231G, G4327A, T5088C, A5270G, C7026T, and G7192A. The complete cDNA sequence of the recombinant Coxsackie virus strain is synthesized by Wuhan Boweid Biotechnology Co., Ltd and constructed onto a eukaryotic expression vector pVAX1 by a molecular biological method, as shown in FIG. 1.

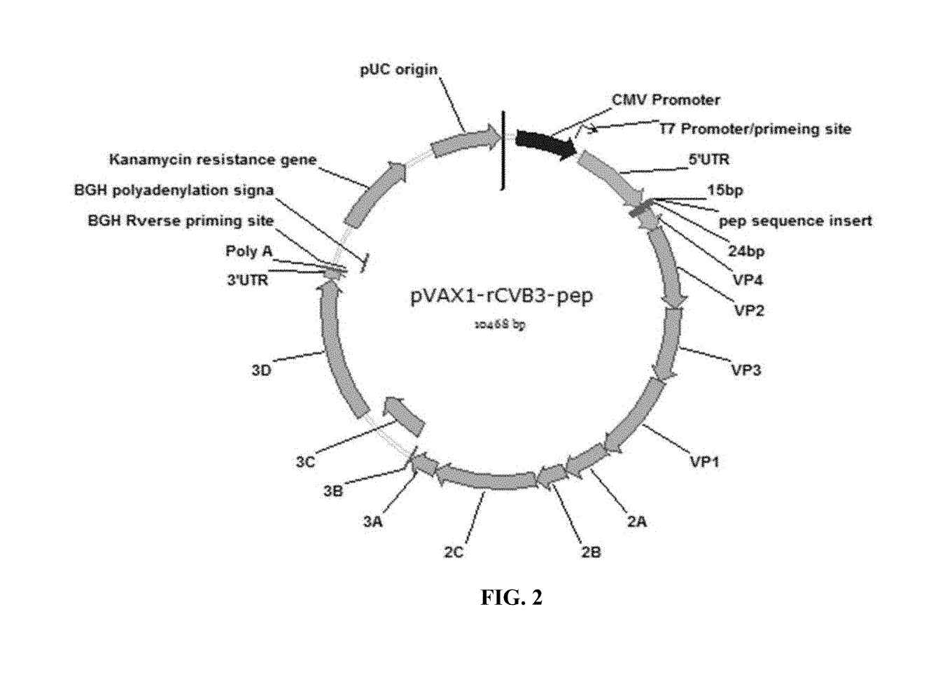

The recombinant oncolytic virus provided in this example comprises the exogenous synthetic DNA sequence inserted between the 5′UTR and VP4 of the constructed recombinant vector by a reverse genetic method. A 15 bp (SEQ ID NO. 1) and a 24 bp (SEQ ID NO. 2) DNA sequence are located at respectively the 5′ and the 3′ terminus of the sequence, which are provided for recognition and cleavage by the Protease C, as shown in FIG. 2.

TABLE 1
Basic peptide fragments and Its Encoding Genes

| | | Polypeptide | | |
|---|---|---|---|---|
| Example | Polypeptide name | sequence | Gene name | Gene sequence |
| 1 | Polypeptide 1 | SEQ ID NO. 3 | nucleotide seque 1 | SEQ ID NO. 4 |
| 2 | Polypeptide 2 | SEQ ID NO. 5 | nucleotide seque 2 | SEQ ID NO. 6 |
| 3 | Polypeptide 3 | SEQ ID NO. 7 | nucleotide seque 3 | SEQ ID NO. 8 |
| 4 | Polypeptide 4 | SEQ ID NO. 9 | nucleotide seque 4 | SEQ ID NO. |
| | | | | 10 |
| 5 | Polypeptide 5 | SEQ ID NO. | nucleotide seque 5 | SEQ ID NO. |
| | | 11 | | 12 |
| 6 | Polypeptide 6 | SEQ ID NO. | nucleotide seque 6 | SEQ ID NO. |
| | | 13 | | 14 |
| 7 | Polypeptide 7 | SEQ ID NO. | nucleotide seque 7 | SEQ ID NO. |
| | | 15 | | 16 |
| 8 | Polypeptide 8 | SEQ ID NO. | nucleotide seque 8 | SEQ ID NO. |
| | | 17 | | 18 |
| 9 | Polypeptide 9 | SEQ ID NO. | nucleotide seque 9 | SEQ ID NO. |
| | | 19 | | 20 |
| 10 | Polypeptide 10 | SEQ ID NO. | nucleotide seque 10 | SEQ ID NO. |
| | | 21 | | 22 |
| 11 | Polypeptide 11 | SEQ ID NO. | nucleotide seque 11 | SEQ ID NO. |
| | | 23 | | 24 |
| 12 | Polypeptide 12 | SEQ ID NO. | nucleotide seque 12 | SEQ ID NO. |
| | | 25 | | 26 |
| 13 | Polypeptide 13 | SEQ ID NO. | nucleotide seque 13 | SEQ ID NO. |
| | | 27 | | 28 |
| 14 | Polypeptide 14 | SEQ ID NO. | nucleotide seque 14 | SEQ ID NO. |
| | | 29 | | 30 |
| 15 | Polypeptide 15 | SEQ ID NO. | nucleotide seque 15 | SEQ ID NO. |
| | | 31 | | 32 |
| 16 | Polypeptide 16 | SEQ ID NO. | nucleotide seque 16 | SEQ ID NO. |
| | | 33 | | 34 |
| 17 | Polypeptide 17 | SEQ ID NO. | nucleotide seque 17 | SEQ ID NO. |
| | | 35 | | 36 |
| 18 | Polypeptide 18 | SEQ ID NO. | nucleotide seque 18 | SEQ ID NO. |
| | | 37 | | 38 |

The sequence is specifically inserted by inserting the basic peptide fragment expressing DNA sequence between the 5′UTR and VP4 of the recombinant vector. After screening, a positive clone is obtained, which is sequenced and extracted for the plasmid, to obtain a complete cDNA for viral packaging.

This example was specifically as follows.

(1) Synthesis of Coxsackie virus gene CVB3-Am

PUC57-CVB3-Am was synthesized by Suzhou Genwiz Biotechnology Co., Ltd.

(2) Mini extraction of vector pVAX1 and pUC19

The pVAX1-SalI and pUC19 plasmids were extracted using the kit available from Axygen.

(3) Construction of pVAX1-SalI-CVB3-Am vector

a. Double cleavage and recovery

The plasmids pVAX1 (Apa I→Sal I) and pUC57-CVB3-Am were cleaved with both NotIand SalI, and subjected to 1% agarose gel electrophoresis after reaction. The vector of 2999 bp and the CVB3-Am fragment of about 7500 bp were recovered, followed by gel extraction. The purification of the cleaved products was carried out following the specific steps as described in instructions for the Gel Extraction Kit from Axygen.

b. Ligation and transformation

The CVB3-Am fragment and the vector pVAX1 (Apa I→Sal I) that were cleaved with both NotIand SalI were ligated at a ratio by the T4DNA ligase available from TAKARA, and then transformed into Stbl3.

c. Screening and identification of positive clones

Single colonies grown on a LB+Kana plate were randomly picked up and subjected to colony PCR. The correct positive clones were sent to Suzhou Genewiz Biotechnology Co., Ltd. for sequencing.

The pVAX1 vector carrying the complete cDNA sequence of the recombinant Coxsackie virus was transfected into Cos7 packaging cells. The cells were cultured to obtain an infectious recombinant virus suspension.

A PolyA sequence ranging from 20 to 100 and preferably from 30 to 80 in length were preferably inserted after the 3′UTR, to effectively ensure the stability of the basic peptide fragment encoding gene, thereby ensuring the expression thereof. The virus can be stored at −20° C. for over 1 year, and at room temperature for 2 days without decline of the titer. Therefore, the virus is highly stable, and convenient in storage and transportation.

Comparative Example 1: The synthetic DNA sequence was inserted between the VP1 and 2A elements in the pVAX1 vector to obtain the virus. The virus could not stably express the basic peptide fragment and had a limited inhibitory effect on cancer cells.

### Example 19. Preparation of Test Samples for Pharmacodynamics Study

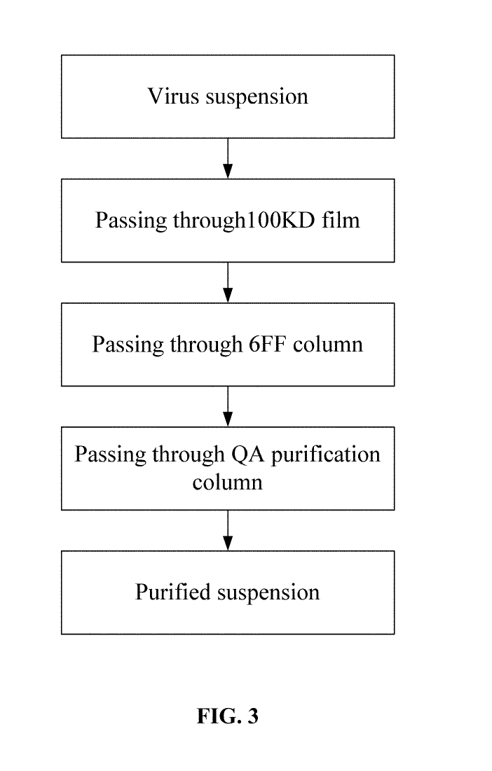

The infectious recombinant virus suspension described in Examples 1 to 18 was inoculated to the expansion cultured Vero cells, and a purified virus suspension was obtained after the production and purification processes, which was used as a test sample. The process for virus purification was as shown in FIG. 3.

The purified virus suspension needs to be inspected to satisfy the following criteria as shown in Table 2.

TABLE 2

| Item | | Standard limit |
|---|---|---|
| of inspection | Method | released |
| Protein | Chinese Pharmacopoeia 2015 Edition, | ≤1 μg/dose |
| content | Part VI, General rule 0731 Protein | |
| | Determination Method V: Bradford | |
| | method. | |
| Virus titer | TCID50 Karber method | ≥10⁷TCID₅₀/mL |

### Example 20. In Vivo Pharmacodynamics Study on the Selective Inhibition of Recombinant Coxsackie Virus on Solid Tumors

The test samples used in this example were prepared and tested according to the protocol described in Example 19.

In this example, the recombinant Coxsackie viruses of Examples 1 to 18 were used as test samples, for example, those prepared in Example 5, Example 14, and Example 17.

The viruses above were prepared into test samples following the method as described in Example 19.

A subcutaneous A549 cell transplanted tumor model of lung cancer in nude mice was established. 30 tumor-bearing animals having uniform tumor volume were screened. The 30 animals with a tumor volume of 45-70 mm³ (average tumor volume of 56 mm³) were assigned to Groups 1-5 at random. Each group of animals were randomly numbered using Excel software and ranked according to the random number from small to large. There were a total of 5 groups, each group having 6 animals. The groups, dosage and administration mode are shown in Table 3.

TABLE 3

| | Agent | | | | |
|---|---|---|---|---|---|
| | administered to | Dosage | Concentration | Administration | Volume |
| Group | the animals | (PFU/kg) | (PFU/mL) | mode | dosed |
| 1 | Saline (negative) | 0.1 mL/10 g | — | Intravenous | 0.1 mL/ |
| | | | | injection | 10 g |
| 2 | Cisplatin | 6 mg/kg | 0.6 mg/mL | Intravenous | 0.1 mL/ |
| | (positive) | | | injection | 10 g |
| 3 | Example 5 | 6 × 10⁶ | 6 × 10⁴ | Intravenous | 0.1 mL/ |
| | | | | injection | 10 g |
| 4 | Example 14 | 6 × 10⁵ | 6 × 10³ | Intravenous | 0.1 mL/ |
| | | | | injection | 10 g |
| 5 | Example 17 | 6 × 10⁴ | 6 × 10² | Intravenous | 0.1 mL/ |
| | | | | injection | 10 g |

The animals in Group 2 (Cisplatin) were administered once a week for 4 consecutive weeks. After 1-week observation, the animals were euthanized on Day 41. In the saline group, the animals were administered with saline once a week for 6 consecutive weeks, and the animals were euthanized on Day 41. The animals were observed twice a day during administration to observe the general clinical symptoms of animals, and the body weight and tumor size were measured twice a week.

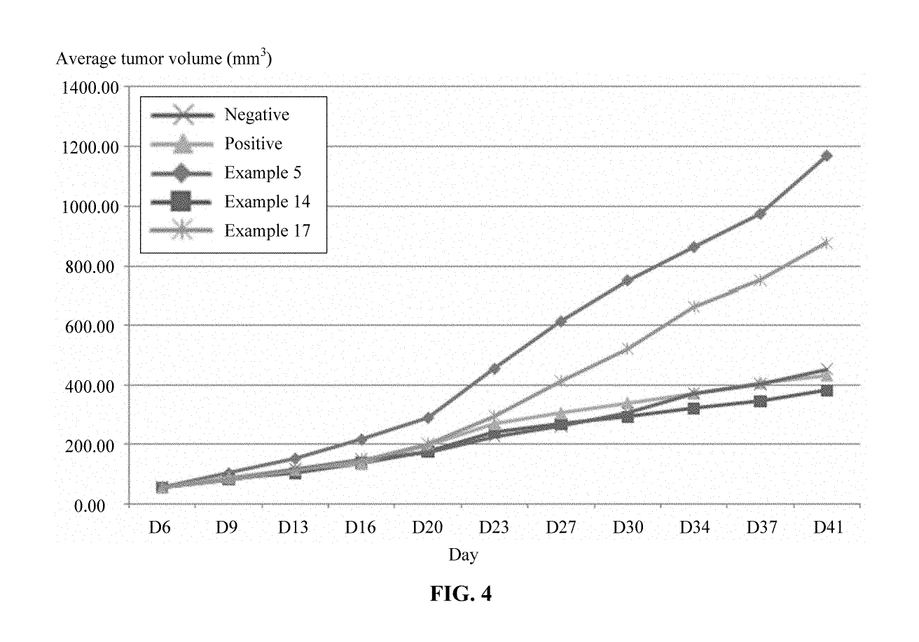

Results: Throughout the experiment, the average body weight of the animals is increased, and there is no significant difference between groups (P<0.05). The curve of increase in average tumor volume of the animals in each group with time is shown in FIG. 4.

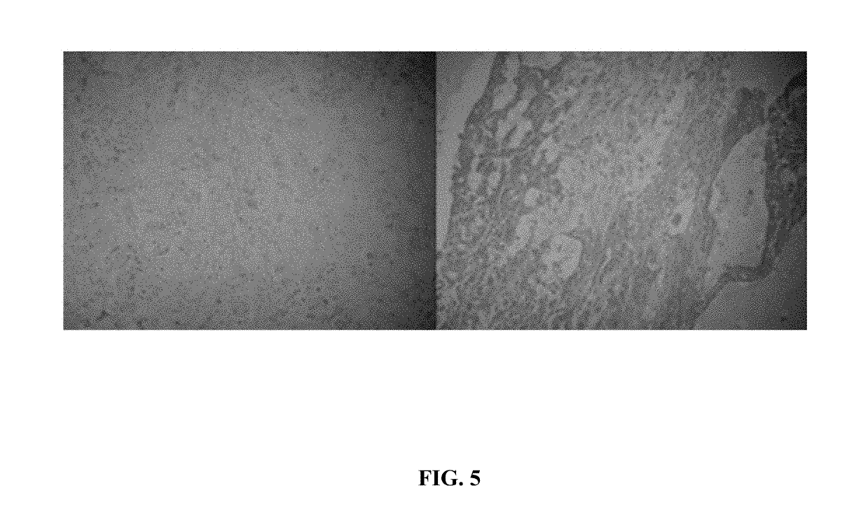

On Day 41, the tissue sections sampled from the animals in the negative control group and in the treatment group with the test sample prepared in Example 5 were examined, and compared, as shown in FIG. 5.

As can be seen from FIG. 5, the treatment group with the test sample prepared in Example 5 on the right has obviously damaged tumor cells and much darker eosin staining, compared with the negative control group on the left.

### Example 21. In Vivo Pharmacodynamics Study on the Selective Inhibition of Recombinant Coxsackie Virus on Solid Tumors

The test samples used in this example were prepared and tested according to the protocol described in Example 19.

In this example, three recombinant Coxsackie viruses having a synthetic DNA sequence inserted in their genomes were used as test samples, which were respectively those prepared in Example 1, Example 2, Example 4, and Example 5.

The viruses above were prepared into test samples following the method as described in Example 19.

A subcutaneous A549 cell transplanted tumor model of lung cancer in nude mice was established. 30 tumor-bearing animals having uniform tumor volume were screened. The 30 animals with a tumor volume of 45-72 mm³ (average tumor volume of 57 mm³) were assigned to Groups 1-6 at random. Each group of animals were randomly numbered using Excel software and ranked according to the random number from small to large. There were a total of 6 groups, each group having 5 animals. The groups, dosage and administration mode are shown in Table 4.

TABLE 4

| | Agent | | | | |
|---|---|---|---|---|---|
| | administered to | Dosage | Concentration | Administration | Volume |
| Group | the animals | (PFU/kg) | (PFU/mL) | mode | dosed |
| 1 | Saline (negative) | 0.1 mL/10 g | — | Intravenous | 0.1 mL/ |
| | | | | injection | 10 g |
| 2 | Cisplatin | 6 mg/kg | 0.6 mg/mL | Intravenous | 0.1 mL/ |
| | (positive) | | | injection | 10 g |
| 3 | Example 1 | 6 × 10⁶ | 6 × 10⁴ | Intravenous | 0.1 mL/ |
| | | | | injection | 10 g |
| 4 | Example 2 | 6 × 10⁵ | 6 × 10³ | Intravenous | 0.1 mL/ |
| | | | | injection | 10 g |
| 5 | Example 4 | 6 × 10⁴ | 6 × 10² | Intravenous | 0.1 mL/ |
| | | | | injection | 10 g |
| 6 | Example 5 | 6 × 10⁴ | 6 × 10² | Intravenous | 0.1 mL/ |
| | | | | injection | 10 g |

The animals in Group 2 (Cisplatin) were administered once a week for 4 consecutive weeks. After 1-week observation, the animals were euthanized on Day 48. In the saline group, the animals were administered with saline once a week for 7 consecutive weeks, and the animals were euthanized on Day 48. The animals were observed twice a day during administration to observe the general clinical symptoms of animals, and the body weight and tumor size were measured twice a week.

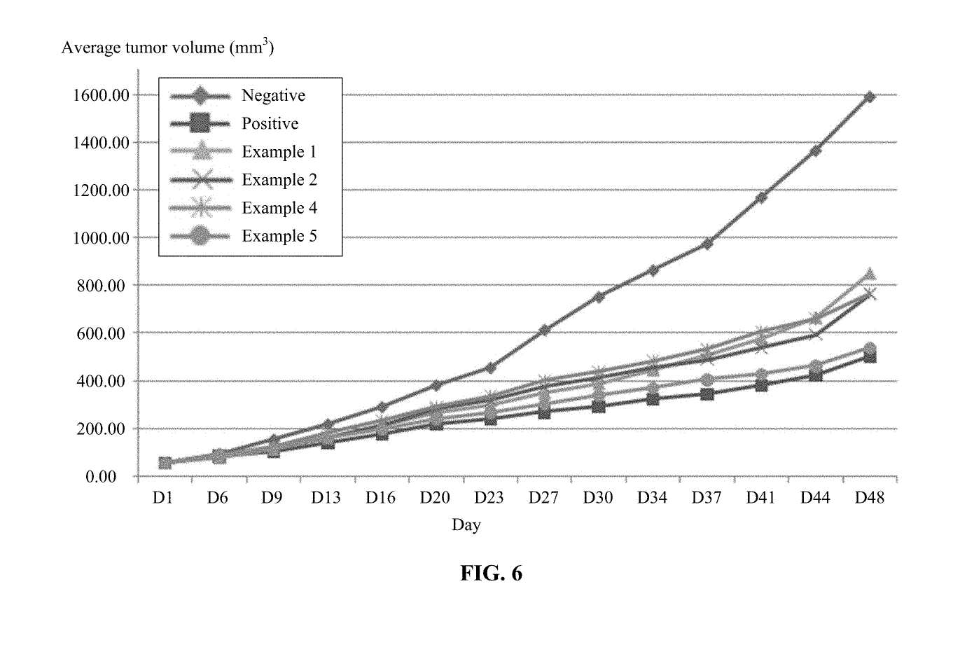

Results: Throughout the experiment, the average body weight of the animals is increased, and there is no significant difference between groups (P<0.05). The curve of increase in average tumor volume of the animals in each group with time is shown in FIG. 6.

It can be seen that the test samples prepared in Examples 1, 2, 4, and 5 all have anti-tumor effects, of which the test sample prepared in Example 5 can significantly inhibits tumor growth.

### Example 22. In Vivo Pharmacodynamics Study on the Selective Inhibition of Recombinant Coxsackie Virus on Solid Tumors

The test samples used in this example were prepared and tested according to the protocol described in Example 19.

In this example, two recombinant Coxsackie viruses having a synthetic DNA sequence inserted in their genomes were used as test samples, which were respectively those prepared in Examples 18, and Example 13.

The viruses above were prepared into test samples following the method as described in Example 19.

A subcutaneous A549 cell transplanted tumor model of lung cancer in nude mice was established. 20 tumor-bearing animals having uniform tumor volume were screened. The 20 animals with a tumor volume of 62-92 mm³ (average tumor volume of 79 mm³) were assigned to Groups 1-4 at random. Each group of animals were randomly numbered using Excel software and ranked according to the random number from small to large. There were a total of 4 groups, each group having 5 animals. The groups, dosage and administration mode are shown in Table 5.

TABLE 5

| | Agent | | | | |
|---|---|---|---|---|---|
| | administered to | Dosage | Concentration | Administration | Volume |
| Group | the animals | (PFU/kg) | (PFU/mL) | mode | dosed |
| 1 | Saline (negative) | 0.1 mL/10 g | — | Intravenous | 0.1 mL/ |
| | | | | injection | 10 g |
| 2 | Cisplatin | 6 mg/kg | 0.6 mg/mL | Intravenous | 0.1 mL/ |
| | (positive) | | | injection | 10 g |
| 3 | Example 18 | 6 × 10⁶ | 6 × 10⁴ | Intravenous | 0.1 mL/ |
| | | | | injection | 10 g |
| 4 | Example 13 | 6 × 10⁵ | 6 × 10³ | Intravenous | 0.1 mL/ |
| | | | | injection | 10 g |

The animals in Group 2 (Cisplatin) were administered once a week for 4 consecutive weeks. After 1-week observation, the animals were euthanized on Day 42. In the saline group, the animals were administered with saline once a week for 6 consecutive weeks, and the animals were euthanized on Day 42. The animals were observed twice a day during administration to observe the general clinical symptoms of animals, and the body weight and tumor size were measured twice a week.

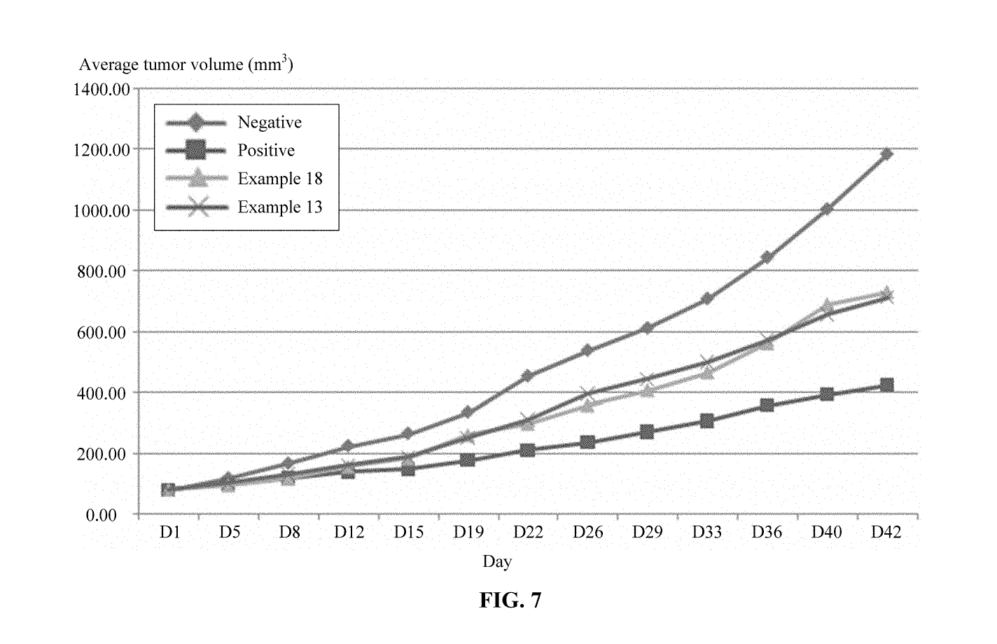

Results: Throughout the experiment, the average body weight of the animals is increased, and there is no significant difference between groups (P<0.05). The curve of increase in average tumor volume of the animals in each group with time is shown in FIG. 7.

It can be seen that the test samples prepared in Examples 18 and 13 both have anti-tumor effects.

### Example 23. In Vivo Pharmacodynamics Study on the Selective Inhibition of Recombinant Coxsackie Virus on Solid Tumors

The test samples used in this example were prepared and tested according to the protocol described in Example 19.

In this example, three recombinant Coxsackie viruses having a synthetic DNA sequence inserted in their genomes were used as test samples, which were respectively those prepared in Examples 8, 9 and 10.

The viruses above were prepared into test samples following the method as described in Example 19.

A subcutaneous A549 cell transplanted tumor model of lung cancer in nude mice was established. 25 tumor-bearing animals having uniform tumor volume were screened. The 25 animals with a tumor volume of 65-90 mm³ (average tumor volume of 79 mm³) were assigned to Groups 1-5 at random. Each group of animals were randomly numbered using Excel software and ranked according to the random number from small to large. There were a total of 5 groups, each group having 5 animals. The groups, dosage and administration mode are shown in Table 6.

TABLE 6

| | Agent | | | | |
|---|---|---|---|---|---|
| | administered to | Dosage | Concentration | Administration | Volume |
| Group | the animals | (PFU/kg) | (PFU/mL) | mode | dosed |
| 1 | Saline (negative) | 0.1 mL/10 g | — | Intravenous | 0.1 mL/ |
| | | | | injection | 10 g |
| 2 | Cisplatin | 6 mg/kg | 0.6 mg/mL | Intravenous | 0.1 mL/ |
| | (positive) | | | injection | 10 g |
| 3 | Example 8 | 6 × 10⁶ | 6 × 10⁴ | Intravenous | 0.1 mL/ |
| | | | | injection | 10 g |
| 4 | Example 9 | 6 × 10⁵ | 6 × 10³ | Intravenous | 0.1 mL/ |
| | | | | injection | 10 g |
| 5 | Example 10 | 6 × 10⁴ | 6 × 10² | Intravenous | 0.1 mL/ |
| | | | | injection | 10 g |

The animals in Group 2 (Cisplatin) were administered once a week for 4 consecutive weeks. After 1-week observation, the animals were euthanized on Day 33. In the saline group, the animals were administered with saline once a week for 5 consecutive weeks, and the animals were euthanized on Day 33. The animals were observed twice a day during administration to observe the general clinical symptoms of animals, and the body weight and tumor size were measured twice a week.

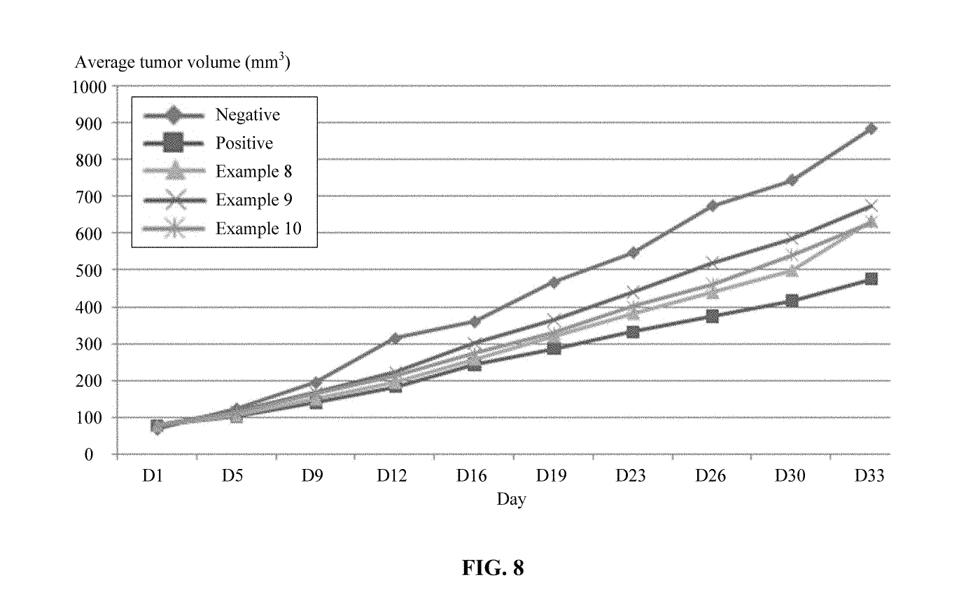

Results: Throughout the experiment, the average body weight of the animals is increased, and there is no significant difference between groups (P<0.05). The curve of increase in average tumor volume of the animals in each group with time is shown in FIG. 8.

It can be seen that the test samples prepared in Examples 8, 9 and 10 both have anti-tumor effects.

The test samples prepared in Examples 1 to 18 all have anti-tumor effects, of which the test samples prepared in Examples 5 and 14 can significantly inhibits tumor growth.

### Example 24. Study on Change of Interstitial pH in Tumors by Recombinant Coxsackie Viruses

In this example, two recombinant Coxsackie viruses having a synthetic DNA sequence inserted in their genomes were used as test samples, which were respectively those prepared in Examples 5, and Example 14.

The two viruses were prepared into test samples following the method as described in Example 19.

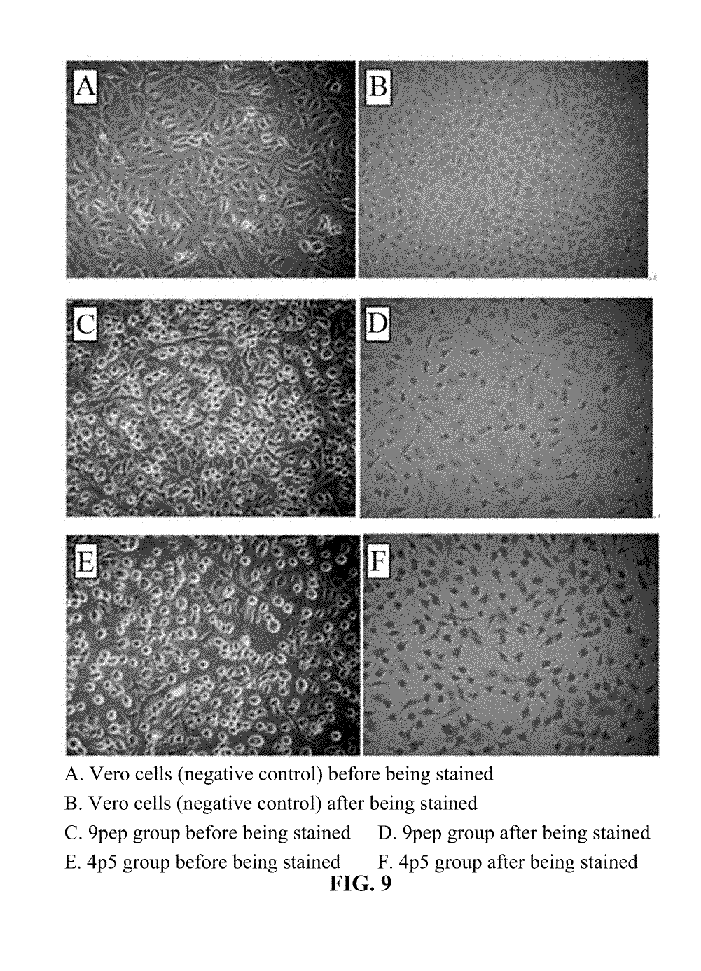

The two test samples were used to infect Vero cells, respectively. The Vero cells infected with the viruses prepared in Example 5 and Example 14 were designated as 4p5 and 9pep groups, respectively. There was another group of cells that was used as a negative control. Each group of cells included two replicates, which were cultured and tested under the same conditions. 3 hrs after infection, one replicate in each group of cells was stained with Eosin and examined microscopically. The result is as shown in FIG. 9.

As can be seen from FIG. 9, the two group of cells infected with the cDNA of recombinant Coxsackie viruses have obvious pathological changes. It can be seen from the staining results that the infection group is stained darker than the negative control group, indicating that its cytoplasm and interstitial space are more acidophilic.

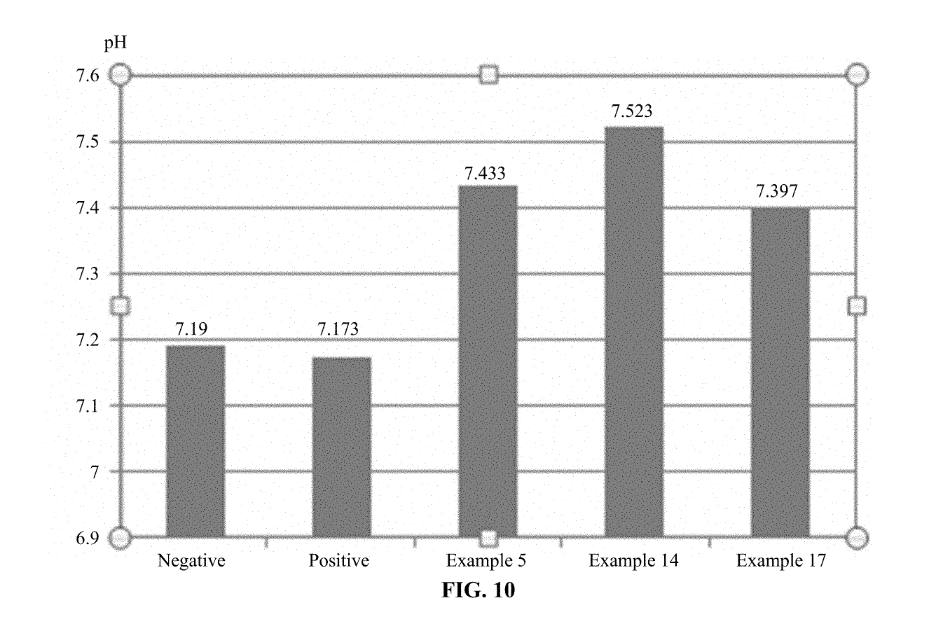

Samples were taken randomly from 3 animals in each test group in Example 20 and the in vivo pH at the tumor site were measured on Day 41 using a Bench-top pH/mV CL-9D02 Meter. The arithmetic mean values of the measurement results for each group are taken, and shown in FIG. 10. The pH value measured with the samples taken from the animals in each test group in Example 20 were found to be increased, by a value of 0.4 to 0.6, as shown in FIG. 10.

It will be obvious to those skilled in the art that changes and modifications may be made, and therefore, the aim in the appended claims is to cover all such changes and modifications.

What is claimed is:

1. A recombinant oncolytic virus, comprising: an oncolytic virus genome and an exogenous DNA sequence inserted in the oncolytic virus genome, the exogenous DNA sequence being adapted to express a basic peptide fragment and to increase an environmental pH in a host infected by the recombinant oncolytic virus.

2. The virus of claim 1, wherein the oncolytic virus genome is a genome of herpes virus, Coxsackie viruses, adenovirus, cowpox virus, measles virus, poliomyelitis virus, retroviruses, reovirus, respiratory syncytial virus, parvovirus H1, vesicular stomatitis virus, or Newcastle disease virus, and preferably adenovirus, Newcastle disease virus, or Coxsackie virus.

3. The virus of claim 1, wherein the environmental pH in the host infected by the recombinant oncolytic virus is increased by about 0.4 to about 0.6.

4. The virus of claim 1, wherein the basic peptide fragment comprises 4 to 10 amino acids.

5. The virus of claim 4, wherein the basic peptide fragment comprises more than 60% of basic amino acids.

6. The virus of claim 5, wherein the basic peptide fragment comprises more than 80% of the basic amino acids.

7. The virus of claim 5, wherein the basic amino acids are selected from arginine, lysine, and histidine.

8. The virus of claim 7, wherein the basic amino acids are selected from arginine and lysine.

9. The virus of claim 1, wherein the basic peptide fragment is selected from:
(SEQ ID NO. 3)
Arg-Lys-Arg-Lys;
(SEQ ID NO. 5)
Lys-Arg-Lys-Arg;
(SEQ ID NO. 7)
Arg-Arg-Lys-Lys;
(SEQ ID NO. 9)
Lys-Lys-Arg-Arg;
(SEQ ID NO. 11)
Lys-Arg-Arg-Lys;
(SEQ ID NO. 13)
Arg-Lys-Lys-Arg;
(SEQ ID NO. 15)
Arg-Arg-His-Lys-Lys;
(SEQ ID NO. 17)
Lys-His-Arg-Lys-His-Arg;
(SEQ ID NO. 19)
Lys-His-Arg-Cys-Lys-Pro;
(SEQ ID NO. 21)
Arg-Arg-His-Lys-Met-Lys;
(SEQ ID NO. 23)
His-Arg-Lys-Cys-Arg-Lys;
(SEQ ID NO. 25)
Lys-Arg-Trp-Arg-Lys-His-Arg;
(SEQ ID NO. 27)
His-Lys-Gly-Arg-Lys-Cys-Arg-Val;
(SEQ ID NO. 29)
Lys-Arg-Trp-His-Lys-Met-Arg-Lys-His;
(SEQ ID NO. 31)
His-Phe-Trp-Arg-Gln-Cys-Ala-Met-Lys;
(SEQ ID NO. 33)
Tyr-Phe-Pro-Arg-His-Gln-Lys-Trp-Lys;
(SEQ ID NO. 35)
Trp-Lys-Tyr-Arg-Gln-Ile-Ser-Thr-Cys;
and
(SEQ ID NO. 37)
Arg-Lys-His-Lys-Met-Arg-Lys-Cys-His-Lys.

10. The virus of claim 1, wherein the recombinant oncolytic virus is Coxsackie virus B3 strain.

11. The virus of claim 10, wherein the basic peptide fragment is selected from:
(SEQ ID NO. 11)
Lys-Arg-Arg-Lys;
(SEQ ID NO. 29)
Lys-Arg-Trp-His-Lys-Met-Arg-Lys-His;
and
(SEQ ID NO. 31)
His-Phe-Trp-Arg-Gln-Cys-Ala-Met-Lys.

12. The virus of claim 11, wherein the recombinant oncolytic virus is a variant attenuated Coxsackie virus B3 strain comprising base mutations of T97C, G1180A, T1654C, T1756C, G2276A, A2685C, G2690A, C3120A, A3231G, G4327A, T5088C, A5270G, C7026T, and/or G7192A.

13. The virus of claim 11, wherein the exogenous DNA sequence is inserted onto a pVAX1 vector.

14. The virus of claim 11, wherein the basic peptide fragment is Lys-Arg-Trp-His-Lys-Met-Arg-Lys-His (SEQ ID NO. 29).

15. A method of preparing an anti-tumor drug comprising employing a recombinant oncolytic virus of claim 1.

16. The method of claim 15, wherein the anti-tumor drug is an anti-solid tumor drug.

17. The method of claim 16, wherein the anti-solid tumor drug is used for treating respiratory tract tumors, gastrointestinal tumors, endocrine tumors, or gynecological tumors.

18. An anti-tumor drug, comprising the recombinant oncolytic virus of claim 1.

19. The drug of claim 18, further comprising a checkpoint inhibitor.

20. A method for treating a malignant tumor, comprising administering the anti-tumor drug of claim 18 intravenously or locally to a patient in need thereof.

21. The method of claim 20, wherein the malignant tumor is a solid tumor.

22. The method of claim 21, wherein the malignant tumor is respiratory tract tumor, gastrointestinal tumor, endocrine tumor, or gynecological tumor.

23. A synthetic DNA sequence, the synthetic DNA sequence being adapted to express a basic peptide fragment comprising more than 60% of basic amino acids.

24. The synthetic DNA sequence of claim 23, wherein the basic peptide fragment comprises more than 80% of the basic amino acids.

25. The synthetic DNA sequence of claim 24, wherein the basic amino acids are selected from Arginine, Lysine, and Histidine.

26. The synthetic DNA sequence of claim 25, wherein the basic amino acids are selected from Arginine, and Lysine.

27. The synthetic DNA sequence of claim 24, wherein the basic peptide fragment is selected from:
(SEQ ID NO. 3)
Arg-Lys-Arg-Lys;
(SEQ ID NO. 5)
Lys-Arg-Lys-Arg;
(SEQ ID NO. 7)
Arg-Arg-Lys-Lys;
(SEQ ID NO. 9)
Lys-Lys-Arg-Arg;
(SEQ ID NO. 11)
Lys-Arg-Arg-Lys;
(SEQ ID NO. 13)
Arg-Lys-Lys-Arg;
(SEQ ID NO. 15)
Arg-Arg-His-Lys-Lys;
(SEQ ID NO. 17)
Lys-His-Arg-Lys-His-Arg;
(SEQ ID NO. 19)
Lys-His-Arg-Cys-Lys-Pro;
(SEQ ID NO. 21)
Arg-Arg-His-Lys-Met-Lys;
(SEQ ID NO. 23)
His-Arg-Lys-Cys-Arg-Lys;
(SEQ ID NO. 25)
Lys-Arg-Trp-Arg-Lys-His-Arg;
(SEQ ID NO. 27)
His-Lys-Gly-Arg-Lys-Cys-Arg-Val;
(SEQ ID NO. 29)
Lys-Arg-Trp-His-Lys-Met-Arg-Lys-His;
(SEQ ID NO. 31)
His-Phe-Trp-Arg-Gln-Cys-Ala-Met-Lys;
(SEQ ID NO. 33)
Tyr-Phe-Pro-Arg-His-Gln-Lys-Trp-Lys;
(SEQ ID NO. 35)
Trp-Lys-Tyr-Arg-Gln-Ile-Ser-Thr-Cys;
and
(SEQ ID NO. 37)
Arg-Lys-His-Lys-Met-Arg-Lys-Cys-His-Lys.

28. The synthetic DNA sequence of claim 27, wherein the basic peptide fragment is selected from:
(SEQ ID NO. 11)
Lys-Arg-Arg-Lys;
(SEQ ID NO. 29)
Lys-Arg-Trp-His-Lys-Met-Arg-Lys-His;
and
(SEQ ID NO. 31)
His-Phe-Trp-Arg-Gln-Cys-Ala-Met-Lys.
